(19) United States
(12) Patent Application Publication
(10) Pub. No.: US 2025/0271262 A1
(43) Pub. Date: Aug. 28, 2025

(54) Method and System for Determining the Total Carbon Balance in a Predetermine Geographic Area

(71) Applicant: Batfer Investment S.A., Montevideo (UY)

(72) Inventors: Diana Carolina Sanchez, Buenos Aires (AR); Adrian Oscar Bucciarelli, Buenos Aires (AR); Mario Ariel Vanzino, Buenos Aires (AR)

(21) Appl. No.: 18/914,547

(22) Filed: Oct. 14, 2024

(30) Foreign Application Priority Data
Feb. 27, 2024 (AR) P20240100445

(51) Int. Cl. G01C 11/04

(52) U.S. Cl. CPC G01C 11/04

(57) ABSTRACT

The invention relates to a method for the determination of the carbon balance in a predetermined geographic area, applicable to natural and/or human-managed systems, which integrates a series of known and validated technologies into a single information system on the carbon sequestered by natural and human-intervened systems. A system for determining the total carbon balance of a predetermined geographic area is also provided.

### CROSS REFERENCE TO RELATED APPLICATION

This application claims priority to Argentine Application No. P20240100445 filed on Feb. 27, 2024, the disclosure of which is incorporated herein by reference for all purposes.

### OBJECT OF THE INVENTION

The present invention refers to a procedure and a system for determining the carbon balance in tons per hectare of a specific geographic area of interest applicable to natural systems, such as native forests and systems modified by man, such as agricultural, farming and forestry systems.

### BACKGROUND OF THE INVENTION

The technical problem associated with determining the carbon balance in a given geographic area has been of interest for the development of economic activities with environmental impact and the use of natural resources in a sustainable manner and has been addressed using different technologies. For example, the document titled “Urban Forestry and Urban Greening”, Volume 67, January 2022, refers to the usefulness of Lansat technology for determining the carbon content sequestered in urban forests and under the title “Quantification of Carbon Sequestration by Urban Forests using Landsat 8 OLI and Machine Learning Algorithms in Jodhpur, India” by Swati Uniyal et al., reveals that urban forests play an important role in the carbon cycle. The quantification of aboveground biomass (AGB, as per its acronym in English) is essential to understand the role of urban forests in carbon sequestration. In this study, machine learning (ML) based regression algorithms (SVM, RF, kNN and XGBoost) have been considered for spatial mapping of AGB and carbon for urban forests of Jodhpur city, Rajasthan, India, with the help of field data and the relations thereof with texture spectra and variables derived from Landsat 8 OLI data.

This document indicates that biomass estimation is affected by many factors where the complexity of the spatially heterogeneous landscape, various environmental factors influencing the distribution of AGB and its upscaling to plot level becomes challenging. Biomass estimation has yielded between 18% and 103% uncertainty due to model-dependent issues, with the plot-level allometric model still the most popular model for AGB estimates.

On the other hand, Chinese application CN114563402 (A) discloses a method for measuring and calculating forest carbon sinks, which includes the following steps: acquiring information on the forest area, land use rate and changing of a region through a remote sensing image, and carrying out a sampling design in a forest; the method comprises the following steps: collecting leaves and soil under the forest in a forest to be detected, and determining the carbon content of plant leaves and the age of each tree in the forest; meanwhile, the organic carbon content in the forest soil is determined; according to the carbon content of plant leaves and the age of each tree in the forest, obtaining an organic carbon multiplication rate, establishing an index multiplication equation, and determining an organic carbon multiplication rate model of trees; detecting the organic carbon content in the soil under the forest and calculating the carbon sequestration capacity of the target forest at a specific tree age; and transmitting the data to a cloud service. According to the method, reliable sample data can be improved, so that the accuracy of forest carbon sink calculation and measurement is improved.

This document combines remote sensing image analysis with soil and vegetation sampling of the forested area where it is desired to determine the sequestered carbon content, among other variables.

The Chinese utility model CN218067826 (U) refers to an integrated agricultural sensor comprising a plurality of MCU modules, where the MCU modules are connected with a plurality of sensor nodes, the MCU modules are connected with a GPRS module, and the sensor nodes comprise a temperature sensor, a humidity sensor, a lighting sensor, a pH value sensor, a carbon dioxide sensor and a soil sensor; the MCU module is connected to the cloud through a router, the router is connected to a support server and a plurality of mobile receiving terminals, and the field crop growth information monitoring system has the technical effect of detecting more comprehensively various crop growth information data in the field.

This document discloses the use of sensor nodes connected to a cloud that allow measuring various soil variables such as pH, humidity, temperature, CO₂ content, and soil sensor to monitor crop growth information.

Application US20140156549 (A1) discloses a method for estimating soil organic carbon (SOC) stocks for a grassland based on plant-derived SOC plus manure-derived SOC minus carbon lost through microbial maintenance respiration. Plant-derived SOC, manure-derived SOC, and microbial maintenance respiration are estimated by considering the effect of one or more of the lignin and cellulose content of the plant material, the estimated annual production of above- and below-ground plants, grazing intensity, the number of fires per year, average annual precipitation, net underground primary production and soil texture. Therefore, the method provides an allocation of plants to compensate for grazing without losing leaf area, and the diversion of carbon through grazing animals and into the soil through the manure deposit.

This document discloses a method for estimating soil organic carbon that considers multiple factors of gain and loss such as those produced by fires and microbial maintenance respiration. The purpose is to compensate for losses due to grazing by growing plants, also considering the carbon contribution through manure. Losses due to fires can be determined through satellite images.

Therefore, there is still a need in the prior art for new and improved methods that reduce the percentage of uncertainty to reasonable values, and consider not only the various soil characteristic variables that correlate with the actual sequestered carbon content of a specific georeferenced area (1), but also consider estimates of the carbon content sequestered in native forests (2) or modified by man (3) present in a predetermined geographic area within which said georeferenced area is located.

Therefore, the present invention provides a method and a system for determining the total balance of carbon sequestered in a predetermined geographic area that considers, in a combined manner, the analysis of satellite images of a predetermined geographic area and the sensorization of soil, climatic, geomorphological and cultural variables which are highly correlated with the carbon sequestration of a specific georeferenced area (1) within said predetermined geographic area, so that it is possible to know remotely and in real time, the total balance of carbon sequestered in said predetermined geographic area. The carbon sequestered in geographic areas with native forests or agricultural or forestry activities can offset the emissions of other intensive economic activities in relation to their emissions (industries such as steel, oil or mining).

### SUMMARY OF THE INVENTION

The present invention refers to a method and a system for determining the carbon balance in a predetermined geographic area, applicable to natural and/or human-managed systems, characterized in that said method comprises the steps of:
A—determining a set of representative attributes of a specific georeferenced area within a predetermined geographic area, by means of at least one measurement station (4) located within said specific georeferenced area (1), where said representative attributes are selected from: soil attributes, climatological attributes, and geomorphological attributes,
B—obtaining soil samples within said specific georeferenced area (1) and determining by physicochemical analysis, the content of sequestered C and the soil and geomorphological attributes corresponding to said soil samples from said specific georeferenced area (1),
C—building a correlation model with the values obtained in step A) for soil, climatological, and geomorphological attributes and with the values obtained in step B) for soil and geomorphological attributes obtained from said specific georeferenced area (1), and then, determining the total sequestered carbon of said specific georeferenced area (1).
D—determining the total sequestered carbon corresponding to the areas of native forests (2) and/or logged forests (3) present within the predetermined geographic area through analysis and processing of satellite images,
E—determining carbon losses caused by the presence of cattle through the estimation of stocking rate (number of heads of cattle per unit area) and a CO₂ emission factor per unit area, and the losses caused by fires through analysis and processing of satellite images within the predetermined geographic area, and
F—estimating the total carbon balance as the difference between the total sequestered carbon obtained in steps C) and d), and the total carbon losses obtained in step E) of the predetermined geographic area.

### BRIEF DESCRIPTION OF THE FIGURES

FIG. 1 shows a schematic view of carbon gains and losses in natural systems.

FIG. 2 shows a schematic view of a grid arrangement of sensor nodes of a measurement station according to the present invention.

FIG. 3 shows a schematic view of the system for determining the carbon balance of a predetermined geographic region.

FIG. 4 plots a distribution scheme in a Sampling Unit.

FIG. 5 is an object-based classification through images.

### DETAILED DESCRIPTION OF THE INVENTION

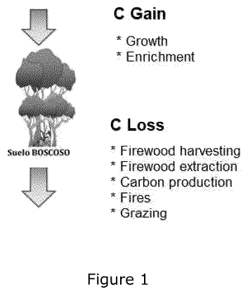

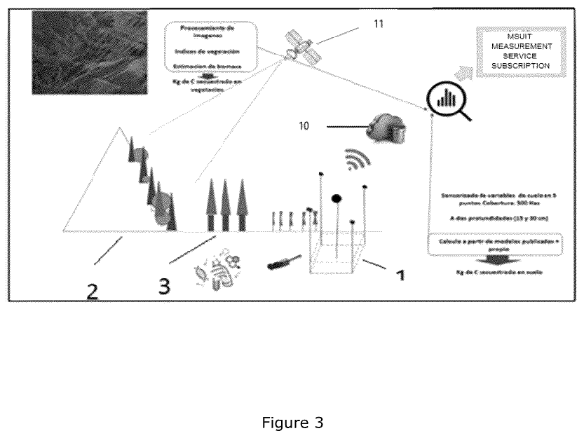

The invention will now be described with reference to FIGS. 1-5.

The present invention provides a method and a system framed within nature-based solutions where carbon is captured and that functionality in the system is increased, through systematic and verifiable measurements for one's own use or to meet the requirements of regulated carbon markets. Through the present invention, it is possible to measure the carbon content sequestered by natural and human-managed systems, thus compensating for the losses produced by industrial, oil or mining operations in a certain geographic area.

Even more so, the method of the present invention eliminates measurement biases because a large part of the procedure is automated and it allows direct transmission of data in real time with the frequency required by the user.

In addition, the present invention allows to adapt, at the local level, information required at the national and regional level for the reports to the nationally determined contributions, where the Nationally Determined Contributions (NDC, for its acronym in English) are the commitments assumed by the countries party to the United Nations Framework Convention on Climate Change (UNFCCC) and that must be carried out to intensify their actions against global climate change, whether to reduce Greenhouse Gas (GHG) emissions (mitigation actions) or to adapt to impacts produced by that phenomenon (adaptation actions). Each country's contributions are established based on their national circumstances and their respective capacities.

The set of NDC contributions submitted by each country should contribute to meeting the objectives of the Paris Agreement of “keeping the global average temperature rise well below 2° C. (35.6° F.) with respect to pre-industrial levels, and continuing efforts to limit this temperature increase to 1.5° C. (34.7° F.) with respect to pre-industrial levels.”

The present invention finds particular application in the categories of activities provided for by the VCS (Verified Carbon Standard) Program for the development of projects and methodologies in Agriculture, Forestry and Other Land Use (AFOLU, as per its acronym in English), including:

Afforestation, Reforestation and Revegetation (ARR) which is one of the six categories that meet the requirements of the “Verified Carbon Standard” activities. This category includes eligible Assisted or Accelerated Natural Regeneration (ANR) activities.
Agricultural Land Management (ALM),
Improved Forest Management (IFM),
Reduced Emissions from Deforestation and Forest Degradation (REDD),
Emissions avoided from preventing the conversion of grasslands and shrublands (ACOGS) and
Wetlands Restoration and Conservation (WRC).

The present invention is based on a carbon gain and loss estimation method (FIG. 1).

Gains in the carbon stock of a system are associated with vegetation growth (above and below the soil surface) and enrichment by the establishment of new specimens. The growth speed will depend on the plant species and the soil and climate conditions.

Losses occur due to vegetation extraction, firewood harvesting, grazing and fires.

In agricultural systems, gains and losses are strongly influenced by cultural practices, which can compensate for the effects of soil type and climate at the regional level.

According to the present invention, to quantify the total balance of carbon sequestered in a predetermined geographic area, it is necessary to consider both the Gains and Losses of the carbon content:

Carbon gains:
Carbon gains are determined based on the following measurements:

### I—Carbon Content Sequestered by the Existing Vegetation Cover Above and Below the Surface:

The cartographic base where the information will be dumped is a geographic information system (GIS). One of the information layers will reflect the carbon sequestered by the vegetation (Vegetation GIS). The quantification of biomass and the variations thereof over time is carried out by analyzing and processing satellite images, for example, obtained through Landsat (11), which allows obtaining the following coverage indexes:
NDVI: Normalized Difference Vegetation Index
EVI: Enhanced Vegetation Index
SAVI: Soil-Adjusted Vegetation Index.

The normalized difference vegetation index (NDVI) is obtained from the ratio of the maximum near infrared (NIR) band reflection and the red band absorption (R), is the most commonly used index, and its range is between −1 to 1 with zero as an approximate value of no vegetation. Negative values represent surfaces without vegetation, while values close to 1 contain dense vegetation; through ArcGis software from a raster calculator extension (ArcGIS geoprocessing tool that runs a raster analysis using a Map Algebra expression), it is calculated using the following equation.

$$NDVI = \frac{NIR - R}{NIR + R}$$

Wherein: NIR: Near Infrared Band
R: Red-Band Reflectance.

The abovementioned information is adjusted by in-field forest inventory from secondary sources (e.g. national forest inventories carried out in the countries to report the progress of the Nationally Determined Contributions to the Paris Agreement) and primary sources at the time of installation of the system, through which a forest inventory is prepared under the national inventory methodology (official). The inventory provides the base information on which the corresponding expansion factors are applied to obtain the aerial tree biomass, carbon stock and carbon sequestration rate through local allometric models that allow estimating these parameters based on a few easily measured variables, such as trunk diameter at breast height (DBH) and/or total height (Loetsch et al. 1973, Caillez 1980, Husch et al. 1982, Parresol 1999), and growth rates per species published on a scientific basis.

### II—Carbon Content Sequestered in Soils:

To estimate carbon sequestered in soil, measurements of soil variables and climatic variables in a specific georeferenced area are used (1). These data can be obtained through conventional physical sampling and laboratory analysis, and/or through automatic sensing of soil attributes and climatic variables using measurement stations that include sensor nodes and at least one weather station for such purposes.

The variables measured for soil attributes, climatological attributes, are the following:
I—Soil attributes: pH, temperature, NPK, electrical conductivity, humidity, texture, bulk density, NPK
II—Climatological attributes: such as rainfall intensity, ambient temperatures, relative humidity, speed and predominant direction of winds, precipitation.
III—Geomorphological attributes: slope (length and degree) and altitude, soil morphology,
IV—Cultivating attributes: tillage methods.

The influence of each attribute on carbon sequestration is described below:

The aforementioned attributes condition the stability of organic carbon in soils and/or have a significant association with soil carbon content.

Numerous studies show the relation between these characteristic attributes with the carbon content, which are incorporated by reference to the present application.

For example, soil pH stabilizes the nutrient pool and soil fertility (Campbell, C. A., B. G. McConkey, R. P. Zentner, F. Selles, D. Curtin, 1996), moisture content presents an association with total organic carbon and with total organic matter; (Azlan A., Aweng E. R., Ibrahim C. O., Noorhaidag, A., 2002), electrical conductivity is related to soil properties and nutrients, (Mauricio Simón et al., July 2013), texture presents a significant association with carbon total organic matter and with total organic matter (Azlan A., Aweng E. R., Ibrahim C. O., Noorhaidag, A., 2002) and (Burke, I. C., C. M. Yonker, W. J. Parton, C. V. Cole, K. Flach, D. S. Schimel, 1989), bulk density allows the quantification of carbon for agronomic soil management (Keith Paustian, et al., 2019), NPK is an indicator of fertility pool, (Alvarez et al., 2012), soil slope (length and degree) is associated with carbon sequestered in soils (Burke, I. C., 1999), altitude (masl) conditions the water balance and the relationship between total organic carbon and soil organic matter (Tan et al., 2004), precipitation shows a significant association with total organic carbon (Azlan A., Aweng E. R., Ibrahim C. O., Noorhaidag, A., 2002) and with organic matter (Alvarez, R., R. S. Lavado, 1998), temperature shows association with organic matter (Alvarez, R., R. S. Lavado, 1998). On the other hand, the tillage method as a cultivating attribute is associated with carbon content (Kong, X. B., T. H. Dao, J. Qin, H. Qin, C. Li, F. Zhang, 2009). Geoderma., 154: 86-92 (2009)).

The total organic carbon content can be estimated from soil organic matter considering a Van Benmelen Factor of 1.724, which results from the assumption that soil organic matter contains 58% Carbon (1/0.58=1.724); (Vela Correa G., López Blanco J., Rodriguez Gamiño L. M. (2011)).

The abovementioned attributes are measured in a specific georeferenced area; therefore, they are georeferenced attributes which allows their incorporation into a layer of the geographic information system (GIS).

Through spatial correlation (e.g., using known statistical data analysis techniques such as Ordinary Least Squares (OLS); autocorrelation analysis) of the values of these georeferenced attributes, a GIS layer of soil carbon is obtained.

### III—Losses of Carbon Content

Carbon content losses are determined based on the following estimates:

### Losses Caused by Cattle:

For this estimation, the stocking rate (number of heads of livestock in a given geographic area) is determined and calculated by a CO₂ emission factor into the atmosphere per unit area (Ha) that represents a measure of the carbon content of the soil that is lost. Preferably, according to the estimated stocking rate, the Intergovernmental Panel on Climate Change (IPCC) emission factor is applied by default.

### Due to Fires: Surface Detectable in Satellite Images

### Losses Caused by Fires:

The surface affected by fires is detectable through satellite images; for the estimation thereof, the surface affected by the fire is calculated and the carbon in soils and vegetation is subtracted for that surface.

Therefore, based on the estimation of carbon gains and losses in soils, the present invention provides a reliable method for determining the total carbon balance of a geographic area applicable to natural and/or human-managed systems, where said method includes the following steps:
A—determining a set of representative attributes of a specific georeferenced area within a predetermined geographic area, by means of at least one measurement station (4) located within said specific georeferenced area (1), where said representative attributes are selected from: soil attributes, climatological attributes, and geomorphological attributes,
B—obtaining soil samples within said specific georeferenced area (1) and determining by physicochemical analysis, the content of sequestered C and the soil and geomorphological attributes corresponding to said soil samples from said specific georeferenced area (1),
C—building a correlation model with the values obtained in step A) for soil, climatological, and geomorphological attributes and the values obtained in step B) for soil and geomorphological attributes and sequestered carbon corresponding to the samples of soil obtained from said specific georeferenced area (1), and determining the total sequestered carbon of said specific georeferenced area (1).
D—determining the total sequestered carbon corresponding to the areas of native forests and/or logged forests present within the predetermined geographic area through analysis and processing of satellite images, preferably, images obtained from LandSAT, for which the following parameters are determined:
   Normalized Difference Vegetation Index (NDVI),
   Enhanced Vegetation Index (EVI),
   Soil Adjusted Vegetation Index (SAVI),
   vegetation cover,
   seasonal variability of the vegetation, and
   vegetation data including cubage, forest species expansion, and crop factors,
all these determined from the bands of satellite images corresponding to three channels: red, green and near infrared,
E—determining the carbon losses caused by the presence of cattle by estimating the stocking rate (number of heads of cattle per unit area) and a CO₂ emission factor per unit area, and the losses caused by fires through analysis and processing of satellite images within the predetermined geographic area,
F—estimating the total carbon balance as the difference between the total sequestered carbon obtained in steps C) and d), and the total carbon losses obtained in step E) of the predetermined geographic area.

In a preferred embodiment of the invention, the representative attributes of the georeferenced area include the following:
soil attributes selected from pH, temperature, electrical conductivity, humidity, texture, bulk density, and NPK,
climatological attributes selected from air temperature, precipitation, relative humidity, wind speed, wind direction, rain intensity, and
geomorphological attributes selected from soil morphology, slope (length and degree) and altitude.

Additionally, cultivation attributes related to soil tillage methods are considered, since they have an influence on the carbon sequestered in the soil.

In another preferred embodiment, step B) of obtaining soil samples within said specific georeferenced area (1) and determining by physicochemical analyses the content of sequestered C and the soil and geomorphological attributes corresponding to said soil samples is carried out by well-known conventional procedures. Such physicochemical analyses can be repeated over time if necessary, for example over periods of at least two years in order to calibrate the models for estimating changes in these attributes over time.

In this regard, it should be noted that changes in such attributes, e.g., soil moisture, have stable correlations with organic matter content, as disclosed in the document titled “Correlation between Soil Organic Matter, Total Organic Matter and Water Content with Climate and Depths of Soil at Different Land use in Kelantan, Malaysia”, Azlan, et al. (2012), the full contents of which are incorporated by reference.

Another object of the invention refers to a system for determining the total carbon balance in a predetermined geographic area comprising:
A) At least one measurement station (4) configured to be installed in a specific georeferenced area within said predetermined geographic area, said at least one measurement station (4) comprising:
a main node (5) and at least one peripheral node (6) arranged in said specific georeferenced area (1),
said main node (5) comprising at least one weather station (9), and at least one soil sensor unit (7), both in communication with control and data transmission means (8) located within the specific georeferenced area, said at least one peripheral node (6) including at least one unit of soil sensors (7) in communication with control and data transmission means (8) located within a specific georeferenced area (1),
B) a remote data reception and processing unit (10) configured to receive and process the data sent from said at least one measurement station (4) within the specific georeferenced area (1) and also to receive, analyze and process satellite images of said predetermined geographic area, and
C) means for determining the sequestered carbon and soil attributes of a plurality of soil samples extracted from said specific georeferenced area (1) within said predetermined geographic area.

In a preferred embodiment, each soil sensor unit (7) comprises a plurality of sensors configured to measure soil variables selected among NPK, temperature, pH, electrical conductivity, and humidity; while the weather station (9) of said at least one measurement station (4) allows determining the climatological variables of said specific georeferenced area, such as, rainfall intensity, ambient temperatures, relative humidity, prevailing wind speed and direction, precipitation and air temperature.

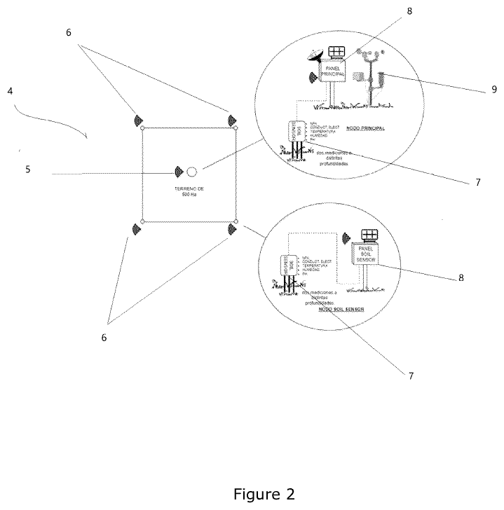

In another preferred embodiment, at least one measurement station (4) comprising a main node (5) and four peripheral nodes (6) preferably arranged in a grid format are arranged within a specific georeferenced area. For example, a preferred format could be a square where at each vertex there is a peripheral node (6) and at the intersection of its diagonals a main node (5) (see diagram in FIG. 2).

Each peripheral node (6) comprises a soil sensor unit (7) including a plurality of soil sensors that are in wired or wireless communication with control and data transmission means. Said control and data transmission means (8) are configured as a peripheral sensor panel located in the vicinity of said sensor unit (7) and comprises data acquisition means that make data transmission possible using an available communication network.

Similarly, the main node (5) includes a soil sensor unit (7) comprising a set of soil sensors configured to measure soil attributes selected from NPK, temperature, pH, electrical conductivity, and moisture, said sensor unit being in wired or wireless communication with control and data transmission means (8) configured as a main sensor panel including data acquisition means that make data transmission possible using an available communication network. Likewise, said main panel of sensors is in communication with a weather station (9) configured to measure rainfall intensity, ambient temperatures, relative humidity, prevailing wind speed and direction, precipitation, and air temperature.

For data transmission, it is possible to use a data transmission network available in the predetermined geographic region. Said network can use the IoT protocol, Lorawan, Wi-Fi, Bluetooth, 4G, 5G, among others.

Each node of a measurement station (4) determines a georeferenced measurement point that allows the information obtained to be integrated into a geographic information system (GIS).

In a preferred embodiment, during step B) of the method of the present invention, the soil sampling point is georeferenced by recording the degree, altitude (slope) and length of the sample extraction point. The bulk density of the sample is also determined, which will allow the volumetric calculation of the C content of the sample.

In a conventional physical-chemical laboratory, all the soil attributes listed above and, additionally, the following soil attributes will be analytically determined on each soil sample extracted in step B):
organic matter content in the soil sample (by Walkley and Black's method consisting of wet oxidation of the soil sample with potassium dichromate in acidic medium. The heat released during the addition of concentrated sulfuric acid is what allows the partial oxidation of C. After a certain time, the mixture is diluted, phosphoric acid is added to avoid interference of Fe3+ and the residual potassium dichromate is titrated with ferrous sulfate).
total organic carbon content,
textural class of the soil according to the percentage of sand, the percentage of silt, the percentage of clay thereof,
concentrations of nitrogen (N), phosphorus (P) and potassium (K), as well as the concentration of cation exchange bases.

With the abovementioned attribute values obtained by known analytical methods, a correlation model is built and by spatial interpolation a GIS (geographic information system) Soil Carbon “shapefile” is obtained for at least two sampling depths (a shapefile is a simple, non-topological format used to store the geometric location and attribute information of geographic entities).

The next step of the method of the present invention is to perform a forest inventory of native forests (2) within the determined geographic area, which is a field activity, where in an area of one hectare in a round plot (which arises from the official native forest inventory methods) each species present is identified, measured with allometric data instruments and then, the basal area and the area of cover are calculated for each species. These values are paired with the information obtained from the analysis and processing of satellite images at the pixel level and allow inference over the entire measurement surface. This methodology is known as “Ground Truth” which allows image data to be related to real features and materials on the ground. Real data collection allows calibration of remote sensing data and assists in the interpretation and analysis of what is being detected.

More specifically, “Ground Truth” can refer to a process in which the “pixels” of a satellite image are compared to what is displayed (at the time of capture) to verify the content of the “pixels” of the image (taking into account that the concept of “pixel” depends on the imaging system). In the case of a classified image, supervised classification can help determine the classification accuracy by the remote sensing system, which can minimize the classification error.

Ground verification is typically performed on site, correlating what is known with surface observations and measurements of various properties of the resolution cell features of the terrain under study in the remotely sensed digital image. The process also involves taking geographic coordinates from the GPS-enabled ground-resolution cell and comparing them to the coordinates of the “pixel” being studied provided by the remote sensing software to understand and analyze location errors and how they can affect a particular study.

According to the present invention, the forest inventory is carried out only on native forests (2) (with or without undergrowth livestock) because in implanted forests (3) (forest plantations), these data are designed according to the species and plantation, i.e., the forest inventory is not applied in agricultural fields.

Preferably, both the distribution of sampling points (specific geo-referenced areas) and the location of forest inventory plots adopt the grid format, with an optimal sampling density determination that considers variability and representativeness with statistical basis at the level of the plot where it is applied.

The distribution and number of inventory plots are calculated and located according to the variance (a measure of the variability of the local vegetation) to ensure representativeness in each particular case.

Preferably, at each georeferenced measurement point, at least two soil samples are taken at different depths, for example, at depths between 0 and 30 cm (11.80 in), preferably between 15 cm (5.90 in) and 30 cm (11.80 in) for the determination of the actual carbon content in the laboratory, and as already indicated, together with the measurement of the surface slope (degree and geographical length) and the altitude (geomorphological attributes) at the measurement points, it is possible to build a correlation model between soil and climatological attributes calculated by the measurement station (4) and the carbon content and soil attributes of the soil samples determined by known laboratory techniques. Then, through spatial interpolation, it is possible to obtain a shapefile in GIS (geographic information system) format of the Soil Carbon content for said two depths of 15 cm (5.90 in) and 30 cm (11.80 in) of said specific georeferenced area.

In another preferred embodiment of the invention, the distribution of the nodes of a measurement station (4) is configured in a grid format and has a coverage area between 400 ha (988.41 acres) and 600 ha (1482.62 acres), preferably, 500 ha (1235.52 acres), with a measurement node every 100 ha (247.10 acres).

The density of measurement stations is adjustable with environmental variability based on the analysis of satellite images and with the viability of the data transmission technology available in the geographic region of interest.

By means of the system and method of the present invention, it is possible to obtain, in real time and remotely, the determination of the total carbon sequestered by the vegetation, the total carbon sequestered by the soil and the total carbon balance captured in a predetermined geographic area, under the format of a shapefile of a geographic information system that can optionally incorporate fire risk alerts.

A generic example of execution of the method of determining the total carbon balance in a predetermined geographic area according to the present invention is described below.

The present method for determining the total carbon balance in a predetermined geographic area applicable to natural and/or human-managed systems according to the present invention as described above, integrates different methodologies for measuring edaphic, climatic, topographic and vegetation variables to obtain total organic carbon values in soils from algorithms obtained in regional studies published at a global level.

Three essential methodologies are used for this purpose:
remote sensing image processing methodologies (analysis and processing of satellite images, for example, Lansat)
traditional methodologies (field soil sampling and forest inventory or “ground truth”)
automatic data capture through georeferenced measurement stations including a plurality of sensors for the determination of soil attributes, at various depths between 0-30 cm [0-11.80 in] deep) as well as climatic and geomorphological attributes.

The method considers carbon gains and losses. This method includes all processes that bring about changes in the carbon pool of a natural system.

Carbon balance can be expressed by the following equation

ΔC=ΔCG−ΔCL
Wherein:
ΔC=annual carbon stock balance of a reservoir, ton C/year.
ΔCG=annual carbon gain, ton C/year
ΔCL=annual carbon loss, ton C/year⁻¹

The calculation of carbon stock applies to any of the AFOLU (Agriculture, Forestry and Other Land Use) categories as established by the IPCC¹ guidelines: ¹IPCC (2006) Guidelines for National Greenhouse Gas Inventories. Chapter 2. Generic Methodologies applicable to multiple land-use categories.

| Land Use and | | |
|---|---|---|
| Management | | |
| Categories | Subcategory | Abbreviation |
| Forest lands | Forest lands that remain as such | FF |
| | Land converted to forest land | LF |
| Cropland | Cropland that remains as such | CC |
| | Land converted to cropland | LC |
| Grasslands | Grasslands that remain as such | GG |
| | Land converted to pasture | LG |
| Settlements | Settlements that remain as such | SS |
| | Land converted to settlements | LS |

Including measurement of:
Aboveground biomass: all biomass of living vegetation, both woody and herbaceous, above ground, including stems, vines, branches, bark, seeds and foliage.
Belowground biomass: all biomass of living roots. Generally, fine roots of less than 2 mm in diameter, are excluded because, empirically, they cannot be distinguished from soil organic matter or leaf litter and
Soil organic matter: includes the organic carbon contained in mineral soils up to a specific depth chosen by the country where it is measured and applied consistently over a period of time. Fine living and dead roots and dead organic matter (DOM) found within the soil and measuring less than the minimum diameter limit (suggested 2 mm) for roots, where DOM is included with soil organic matter when it cannot be distinguished from the latter empirically.

The default value for soil depth is 30 cm (11.80 in) and each country establishes guidelines on how to determine specific depths for determining soil carbon content.

Steps of the method for calculating the total carbon balance:
a) Determining a set of attributes representative of a specific georeferenced area (1) within a predetermined geographic area, by means of at least one measurement station located within said specific georeferenced area, wherein said representative attributes include:
I—Automatic measurements of the following climatological attributes carried out by the georeferenced weather station of said measurement station:
I—Air temperature
II—Precipitation
III—Humidity
IV—Wind speed
V—Wind direction
(since these variables vary over time, the frequency of data recording is statistically adjusted)

The values between each of the weather stations are integrated with spatial correlation methods.

Measurements of the following soil attributes are obtained from remote measurements of soil sensors at each measurement station:
I—Soil temperature at 10 cm (3.93 in) depth
II—Soil temperature at 30 cm (11.80 in) depth
III—Humidity
IV—pH
V—Electrical conductivity
VI—NPK
(These attributes vary over time; the frequency of data capture must be adjusted statistically).

The geomorphological attributes including slope (length and degree) and altitude and soil morphology are also determined.

The morphology of the soil is obtained by extracting soil samples in situ in the place where the measurement station (4) is installed, with which it is possible to determine the following soil attributes:
% sand at 0-30 cm (0-11.80 in) depth
% silt at 0-30 cm (0-11.80 in) depth
% clay at 0-30 cm (0-11.80 in) depth
Bulk density at 0-30 cm (0-11.80 in) depth

These attributes do not have high variations over time because they are local characteristics of the soil.

B) the sequestered C content and the soil attributes corresponding to these soil samples from this specific georeferenced area (1) are determined by means of physicochemical analyses performed on these samples extracted in situ at depths between 0 and 30 cm (11.80 in).

For example, the indirect determination of organic carbon in soils is possible based on the following attributes measurable at depths from 0 to 30 cm (11.80 in).

$$\% CO 0 - 30 cm ( 0 - 11.8 in ) = ( 0.01 ( \% ARC 0 - 30 cm [ 0 - 11.8 in ] ) + 0.012 ( \% ARE 0 - 30 cm [ 0 - 11.8 in ] ) - DA ) / 0.087$$

Wherein
% CO 0-30 cm (0-11.80 in): Percentage of organic carbon in a soil depth of 0 to 30 cm (11.80 in).
% ARC 0-30 cm (0-11.80 in): Percentage of clay in a soil depth of 0 to 30 cm (11.80 in).
% ARE 0-30 cm (0-11.80 in): Percentage of sand in a soil depth of 0 to 30 cm (11.80 in).
DA: Bulk Density in Kg/m³.
(Source: INTA (Argentine National Institute of Agricultural Technology) “Mapa de almacenamiento de Carbono en los Suelos de la República Argentina” (Carbon storage map in the Soils of the Argentine Republic), 2023).
C—a correlation model is then constructed with the values obtained in step A) for the soil, climatological, and geomorphological attributes and the values obtained in step B) for the soil and sequestered carbon attributes corresponding to the soil samples obtained from that specific georeferenced area (1), and then, determining the total sequestered carbon of that specific georeferenced area (1).
D—determining the total sequestered carbon corresponding to the areas of native forests (2) and/or logged forests (3) present within the predetermined geographic area through analysis and processing of LANSAT satellite images.

Through the analysis and processing of LANSAT satellite images, the values of the topographic attributes for a given georeferenced area are determined:
I—Altitude above sea level
II—Slope gradient
III—Slope length
IV—North orientation degrees
V—South orientation degrees
VI—Shape Index (Gaussian terrain shape classification)

Also, through the analysis and processing of satellite images, the following land cover indexes are calculated:
NDVI: Normalized Difference Vegetation Index
EVI: Enhanced Vegetation Index
SAVI: Soil-Adjusted Vegetation Index.
vegetation cover, seasonal variability of vegetation, and vegetation data including cubage, forest species expansion, and crop factors.

In the case of land covered by native forests, a forest inventory is additionally carried out on a number of plots established by known statistical methods (the greater the variability, the greater the number of these samples). Each country has an established method for developing forest inventories to report nationally determined contributions (NDCs) to the Paris Agreement.

By correlating the data obtained in the field with the vegetation indexes obtained through the analysis and processing of satellite images, it is possible to reduce the frequency of this inventory (i.e. the need for field measurements) and its intensity (i.e. the number of plots to be visited in a given period of time (years), and to carry out field measurements).

For example, in Argentina, the design of the forest inventory must be systematic on a grid of equidistant points. The grid will be generated in the Gauss-Kruger cartographic projection system, zone 4, Datum WGS84. Each grid point corresponds to a Sampling Unit (SU).

Shape and size of the sampling unit (SU).

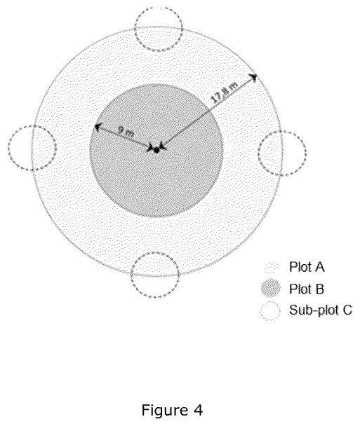

Each SU shall be made up of two circular and concentric plots, designated with the letters A and B, where the woody individuals will be surveyed; and four subplots, designated with the letter C, for surveying the regeneration of woody species.
Plot A shall have an area of 1000 m² (17.8 meter radius)
Plot B shall have an area of 255 m² (9 meter radius)
Plots C shall have a surface area of 12.5 m² (2 meter radius) and shall have the centers thereof arranged 17.8 m from the center of plot A, on the north, south, east and west tangents as shown in the diagram of the FIG. 4.

Measurements and Records of the following data for the preparation of the forest inventory are then carried out:

### From the Sampling Unit (SU):

Identification (ID) of the sampling unit (SU);
Grid coordinates;
SU central coordinates;
Reference point coordinates;
Access reference data;
Observations.

### From Woody Specimens:

Registry number;
Azimuth and distance to the plot center of reference specimens;
Species;
Total height;
Crown diameter (for forest only);
Status of woody specimens;
Number of shafts;
DBH (Diameter at breast height);
DAB (Basal area diameter, for forest only);
Shaft length;
Shaft health;
Shaft shape;
Regeneration (quantity and species).

### From the Site:

Type of landscape;
Altitude;
Slope;
Exposure;
Erosion;
Salinity;
Life forms;
Vegetation covers;
Fallen woody material.

### From Anthropogenic Activities:

Fires;
Grazing/livestock;
Logging.

This information shows that for each pixel (minimum homogeneous unit in a satellite image), there are topographic, climatic, vegetation and edaphic data in different layers of a geographic information system (GIS).

Additionally, the classification of remote sensing data can be based on objects or pixels. In object-based classification, the image is divided into objects using shape and color information, which are then classified using spectral and texture information within each object.

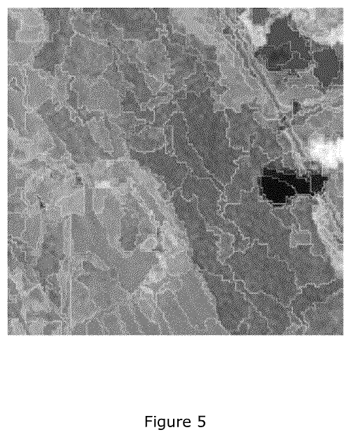

In this type of classification, the software produces lines of the boundaries between objects, efficiently obtaining a result similar to the visual interpretation. For example, FIG. 5 shows an object-based classification using satellite images.

From these data, the algorithms for the relationship between the variables of the different attributes are applied as explained in the preceding description, as they arise from the scientific publications listed below to determine the carbon gains and carbon losses:

The background of practical applications and correlation models between the various soil attribute variables from which the algorithms for the calculation of total organic carbon in soils are taken can be found in the following publications, the complete contents of which are incorporated into the present invention:
Burke, I. C., C. M. Yonker, W. J. Parton, C. V. Cole, K. Flach, D. S. Schimel, 1989. Texture, climate, and cultivation effects on soil organic matter content in US grassland soils. Soil Science Society of America Journal, 53: 800-805
Campbell, C. A., B. G. McConkey, R. P. Zentner, F. Selles, D. Curtin, 1996. Tillage and crop rotation effects on soil organic C and N in a coarse-textured typic Haploboroll in southwestern Saskatchewan. Soil Tillage Res., 37: 3-14
Alvarez, R., R. S. Lavado, 1998. Climate, organic matter and clay content relationships in the Pampa and Chaco soils, Argentina. Geoderma, 83: 127-141.
Burke, I. C., 1999. Spatial variability of soil properties in the short grass steppe: the relative importance of topography, grazing, microsite, and plant species in controlling spatial patterns. Ecosystems, 2: 422-438.
IPCC (2006) Guidelines for National Greenhouse Gas Inventories. Chapter 2. Generic Methodologies applicable to multiple land-use categories.
Kong, X. B., T. H. Dao, J. Qin, H. Qin, C. Li, F. Zhang, 2009. Effects of soil texture and land use interactions on organic carbon in soils in North China cities' urban fringe. Geoderma, 154: 86-92.
Vela Correa G., López Blanco J., Rodriguez Gamiño L. M. (2011) Niveles de carbono orgánico total en el Suelo de Conservación del Distrito Federal, centro de Mexico [“Total Organic Carbon Levels in the Conservation Soil of the Federal District, central Mexico” ]. Investigaciones Geogreficas, Boletin del Instituto de Geografia, UNAM ISSN 0188-4611, No. 77, 2012, pages 18-30
Azlan A., Aweng E. R., Ibrahim C. O., Noorhaidag, A. 2012. Correlation between Soil Organic Matter, Total Organic Matter and Water Content with Climate and Depths of Soil at Different Land use in Kelantan, Malaysia. J. Appl. Sci. Environ. Manage. Vol. 16 (4) 353-358.
Hijmans, R. J.; Cameron, S. E.; Parra, J. L.; Jones, P. G.; Jarvis, A. 2005. Very high resolution interpolated climate surfaces for global land areas. Int J Climatol 25: 1965-78.
Keith Paustian, Sarah Collier, Jeff Baldock, Rachel Burgess, Jeff Creque, Marcia DeLonge, Jennifer Dungait, Ben Ellert, Stefan Frank, Tom Goddard, Bram Govaerts, Mike Grundy, Mark Henning, R. César Izaurralde, Mikuláš Madaras, Brian McConkey, Elizabeth Porzig, Charles Rice, Ross Searle, Nathaniel Seavy, Rastislav Skalsky, William Mulhern & Molly Jahn (2019): Quantifying carbon for agricultural soil management: from the current status toward a global soil information system, Carbon Management, DOI: 10.1080/17583004.2019.1633231
Wiesmeier M., Urbanski L., Hobley E., Lang B., von Lutzow M., Marin-Spiotta E., van Wesemael B., Rabot E., LieB M., GarciaFranco N., Wollschläger U., Vogel H. J., Kögel-Knab I. (2019) Soil organic carbon storage as a key function of soils—A review of drivers and indicators at various scales. Geoderma. Vol 33. January 2019, pages 142-169
Ledesma, S. G.; Sione, S. M. J.; Oszust, J. D.; Rosenberger, L. J. 2020. Estimación del contenido y captura potencial de carbono en la biomasa arbórea de bosques nativos del Espinal [“Estimation of Carbon Content and Potential Carbon Sequestration in the Tree Biomass of Native Forests of the Espinal” ]. (Entre Rios, Argentina). FAVE—Ciencias Agrarias 20 (1): 331-345. CC BY—NC-SA 4.0
Vangi E., D'Amico G., Francini S., Borghi C., Giannetti F., Corona P., Marchetti M., Travaglini D., Pellis G., Vitullo M., Chirici G. (2023) Large-scale high resolution yearly modeling of forest growing stock volume and above-ground carbon pool.
INTA (2023). Mapa de almacenamiento de C en los suelos de la República Argentina [“C Storage Map in the Soils of the Argentine Republic” ]. Asociación Argentina de Productores en Siembra Directa (Aapresid), Consorcio Regional de Experimentación Agricola (CREA), Instituto Nacional de Tecnologia Agropecuaria (INTA), Secretaria de Agricultura, Ganaderia y Pesca de la Nación. Argentina.

Finally, the total carbon balance is determined as the difference between the total carbon sequestered and the total carbon losses of the predetermined geographic area.

1. A method for determining the carbon balance in a predetermined geographic area, applicable to natural and/or human-managed systems, characterized in that said method comprises the steps of:
A—determining a set of representative attributes of a specific georeferenced area (1) within a predetermined geographic area, by means of at least one measurement station (4) located within said specific georeferenced area (1), wherein said representative attributes are selected from: soil attributes, climatological attributes, and geomorphological attributes,
B—obtaining soil samples within said specific georeferenced area (1) and determining by physicochemical analysis, the content of sequestered carbon and the soil and geomorphological attributes corresponding to said soil samples from said specific georeferenced area (1),
C—building a correlation model with the values obtained in step A) for soil, climatological, and geomorphological attributes and the values obtained in step B) for soil and geomorphological attributes and sequestered carbon corresponding to the samples of soil obtained from said specific georeferenced area (1), and determining the total sequestered carbon of said specific georeferenced area (1),
D—determining the total sequestered carbon corresponding to the areas of native forests (2) and/or logged forests (3) present within the predetermined geographic area through analysis and processing of LANSAT satellite images, and through a forest inventory within that predetermined geographic area,
E—determining the carbon losses caused by the presence of cattle by estimating the stocking rate (number of heads of cattle per unit area) and a CO₂ emission factor per unit area, and the losses caused by fires through analysis and processing of satellite images within the predetermined geographic area, and through a forest inventory within that predetermined geographical area,
F—estimating the total carbon balance as the difference between the total sequestered carbon obtained in steps C) and D), and the total carbon losses obtained in step E) of the predetermined geographic area.

2. The method according to claim 1, characterized in that the representative attributes determined in step A) are selected from:
soil attributes selected from pH, temperature, electrical conductivity, humidity, texture, bulk density, and NPK,
climatological attributes: such as rainfall intensity, ambient temperatures, relative humidity %, speed and predominant direction of winds,
geomorphological attributes selected from soil morphology slope (length and degree) and altitude.

3. The method according to claim 1, characterized in that the representative attributes determined in the soil samples obtained in step B) comprise the following:
pH, temperature, electrical conductivity, humidity, texture, bulk density, and NPK,
organic matter content, total organic carbon content,
soil morphology including soil textural class according to the percentage of sand, the percentage of silt, and the percentage of clay thereof,
nitrogen (N), phosphorus (P) and potassium (K) concentrations, as well as the concentration of cation exchange bases.

4. The method according to claim 1, characterized in that the soil samples obtained in step B), are obtained at depths between 0-30 cm (0-11.80 in).

5. The method according to claim 1, characterized in that step D) comprises the analysis and processing of satellite images (LandSAT), whereby the following parameters are determined:
Normalized Difference Vegetation Index (NDVI),
Enhanced Vegetation Index (EVI),
Soil Adjusted Vegetation Index (SAVI),
Vegetation cover,
Seasonal variability of the vegetation, and
Vegetation data including cubage, forest species expansion, and crop factors.

6. The method according to claim 1, characterized in that in step D) for determining total carbon in soils, cultural attributes that are related to land use (whether forestry, agricultural or livestock) are considered.

7. The method according to claim 1, characterized in that the physicochemical analyzes carried out on the soil samples of step B) can be repeated over time.

8. The method according to claim 1, characterized in that in step B), the degree and altitude (slope) and length of the extraction point of the soil sample for georeferencing are recorded.

9. The method according to claim 1, characterized in that the samples of step B) are obtained at two different depths.

10. The method according to claim 9, characterized in that in the samples obtained in step B), the organic carbon in soils is determined according to the following formula:
% CO 0-30 cm (0-11.80 in)=(0.010 (% ARC 0-30 cm [0-11.80 in])+0.012 (% ARE 0-30 cm [0-11.80 in])−DA)/0.087
wherein:
% CO 0-30 cm (0-11.80 in): Percentage of organic carbon in a soil depth of 0 to 30 cm (11.80 in).
% ARC 0-30 cm (0-11.80 in): Percentage of clay in a soil depth of 0 to 30 cm (11.80 in).
% ARE 0-30 cm (0-11.80 in): Percentage of sand in a soil depth of 0 to 30 cm (11.80 in).
DA: Bulk Density in Kg/m³.

11. The method according to claim 1, characterized in that step D) further comprises carrying out a forest inventory by plots to estimate the basal area and coverage area for each native forest species (2).

12. The method according to claim 1, characterized in that the distribution of the soil sampling points in the specific georeferenced areas as well as the location of the forest inventory plots adopt the grid format.

13. A remote system for determining the total carbon balance in a predetermined geographical area according to the method of claim 1, characterized in that it comprises:
A) At least one measurement station (4) configured to be installed in a specific georeferenced area (1) within said predetermined geographic area, said at least one measurement station (4) comprising:
a main node (5) and at least one peripheral node (6) arranged in said specific georeferenced area (1),
said at least one main node (5) comprising at least one weather station (9), and at least one soil sensor unit (7) both in communication with respective control and data transmission means (8) located within the specific georeferenced area (1),
said at least one peripheral node (6) including at least one soil sensor unit (7) in communication with respective control and data transmission means (8) located within a specific georeferenced area (1),
B) a remote data reception and processing unit (10) configured to receive and process the data sent from said at least one measurement station (4) within the specific georeferenced area (1) and also to receive, analyze and process satellite images of said predetermined geographic area, and
C) means for determining the sequestered carbon and soil attributes of a plurality of soil samples extracted from the installation site of the measurement station located within that specific georeferenced area (1).

14. The system according to claim 13, characterized in that each soil sensor unit (7) comprises a plurality of sensors configured to measure soil variables selected from pH, temperature, electrical conductivity, moisture, texture, bulk density, and NPK.

15. The system according to claim 13, characterized in that the weather station (9) of said at least one measurement station (4) is configured to measure climatological variables of said specific georeferenced area (1) selected from rainfall intensity, ambient temperatures, relative humidity %, prevailing wind speed and direction, precipitation and air temperature.

16. The system according to claim 13, characterized in that at least one measurement station (4) comprising a main node (5) and four peripheral nodes are arranged within the specific georeferenced area (1).

17. The system according to claim 16, characterized in that said main node (5) and said four peripheral nodes are distributed in a grid format.

18. The system according to claim 13, characterized in that each soil sensor unit (7) of said main nodes and said peripheral nodes comprises a plurality of soil sensors configured to measure soil attributes selected from NPK, temperature, pH, electrical conductivity, and moisture.

19. The system according to claim 13, characterized in that said control and data transmission means (8) are configured as a sensor panel comprising data acquisition means for data transmission through an available communication network.

20. The system according to claim 13, characterized in that said main sensor panel is in communication with a weather station (9) configured to measure rainfall levels, ambient temperatures, relative humidity, prevailing wind speed and direction, precipitation, and air temperature.

21. The system according to claim 13, characterized in that each node of a measurement station (4) determines a georeferenced measuring point.

22. The system according to claim 13, characterized in that the distribution of the nodes of a measurement station (4) has a coverage area between 400 ha (988.41 acres) and 600 ha (1482.62 acres), with a measurement node every 100 ha (247.10 acres).

23. The system according to claim 13, characterized in that the density of measurement stations (1) is adjustable with environmental variability based on analysis of satellite images.
